(19) United States
(12) Patent Application Publication
(10) Pub. No.: US 2023/0002750 A1
(43) Pub. Date: Jan. 5, 2023

(54) PREPARATION OF ADIPOSE TISSUE-DERIVED CELL POPULATION

(71) Applicants: AMANO ENZYME INC., Nagoya (JP); JICHI MEDICAL UNIVERSITY, Tokyo (JP)

(72) Inventors: Kazuhiro FURUKAWA, Nagoya (JP); Yuki ISHIGAKI, Kakamigahara (JP); Kotaro YOSHIMURA, Shimotsuke (JP); Takako SHIRADO, Shimotsuke (JP); Natsumi SAITO, Shimotsuke (JP); Rintaro ASAHI, Shimotsuke (JP); Masanori MORI, Shimotsuke (JP)

(73) Assignees: AMANO ENZYME INC., Nagoya (JP); JICHI MEDICAL UNIVERSITY, Tokyo (JP)

(21) Appl. No.: 17/782,531

(22) PCT Filed: Dec. 3, 2020
(86) PCT No.: PCT/JP2020/045005

§ 371 Date: Jun. 3, 2022

(30) Foreign Application Priority Data
Dec. 4, 2019 (JP) 2019-219192

(51) Int. Cl. C12N 9/52; C12N 5/071; C12N 5/077

(52) U.S. Cl. CPC C12N 9/52; C12N 5/069; C12Y 304/24027; C12Y 304/24003; C12Y 304/22008; C12N 5/0653; C12N 2509/00

(57) ABSTRACT

A method for preparing a stromal vascular fraction from an adipose tissue, including a step of treating an adipose tissue with an enzyme solution containing a collagenase and a neutral protease, preferably an enzyme solution free of clostripain and thermolysin, and showing not less than 1 U neutral protease activity with respect to 10,000 U collagenase activity, and recovering cells is provided by the present invention.

### TECHNICAL FIELD

The present invention relates to a method for preparing a cell population derived from an adipose tissue, and an enzyme preparation and the like used for the method.

### BACKGROUND ART

Adipose tissue contains mesenchymal stem cells, various stromal cells and progenitor cells thereof, and the like, and is promising as a source (cell source) for regenerative medicine. In addition, adipose tissues are more easily collected than blood, bone marrow, and the like and the utility value thereof is high. In fact, many research groups and research institutions are attempting to utilize mesenchymal stem cells and stromal vascular fraction (SVF) derived from adipose tissue for regenerative medicine.

Vascular endothelial cells (endothelial cell: EC)/vascular endothelial progenitor cells (endothelial progenitor cell: EPC) are themselves useful as transplant materials in regenerative medicine. In addition, they are also useful as a therapeutic tool in regenerative medicine because they can be expected to improve the therapeutic effect in transplantation treatment and the like when used in combination. Enzymes are generally used to prepare SVF containing vascular endothelial cells/vascular endothelial progenitor cells from an adipose tissue. For dispersing adipose tissue, that is, enzymatic disintegration of adipose tissue, for example, an enzyme preparation in which a protease is mixed with collagenase (crude collagenase preparation, for example, collagenase contaminated with Clostripain, Neutral protease, etc.) is used. In addition, collagenase and thermolysin may be used in combination, and an enzyme preparation for such enzymatic disintegration (containing collagenase and thermolysin) is also commercially available (e.g., Liberase manufactured by Roche and Celase manufactured by Cytori therapeutics).

### CITATION LIST

### Patent Literature

### [PTL 1]

JP-A-2019-88279

### SUMMARY OF INVENTION

### Technical Problem

Thermolysin used for the preparation of SVF is known to greatly damage to cells and affects the activity of cells in SVF. While an efficient preparation method has been proposed (see, for example, Patent Literature 1), it is difficult to easily and efficiently prepare high-purity vascular endothelial cells/vascular endothelial progenitor cells (for convenience of explanation, “vascular endothelium-related cell” is used below as a term encompassing vascular endothelial cells and vascular endothelial progenitor cells) from an adipose tissue. In view of the future development of regenerative medicine, it is desired to more efficiently prepare SVF containing vascular endothelium-related cells derived from adipose tissue. Therefore, it is an object of the present invention to provide an effective means for efficient preparation of SVF (in other words, improvement of yield). It is also an object of the present invention to increase the number of vascular endothelium-related cells in SVF and to prepare high-purity vascular endothelium-related cells easily and efficiently.

### Solution to Problem

In the course of studies in view of the above-mentioned problems, the present inventors considered that the conditions for enzymatic disintegration of adipose tissue are the most important for the efficient preparation of SVF, focused on the enzymes to be used, and studied them in detail. As a result, it was found that the combined use of collagenase and neutral protease is particularly effective, and that the ratio of the both enzymes is important. As a result, they have successfully improved the yield of SVF and increased the number of vascular endothelium-related cells in SVF. Based on this achievement, the following inventions are provided.

[1] A method for preparing a stromal vascular fraction from an adipose tissue, the method comprising the following step (1):

(1) a step of treating an adipose tissue with an enzyme solution comprising a collagenase and a neutral protease, and showing not less than 1 U neutral protease activity with respect to 10,000 U collagenase activity, and recovering cells.

[2] The method of [1], wherein the neutral protease activity of the enzyme solution is not less than 2 U with respect to 10,000 U collagenase activity.
[3] The method of [1], wherein the neutral protease activity of the enzyme solution is not less than 2.5 U with respect to 10,000 U collagenase activity.
[4] The method of [1], wherein the enzyme solution has a collagenase:neutral protease activity ratio of from 34,000:9 to 34,000:45.
[5] The method of any one of [1] to [4], wherein the enzyme solution has a collagenase content of not less than 500 U per 1 g of the adipose tissue.
[6] The method of any one of [1] to [4], wherein the enzyme solution has a neutral protease content of not less than 0.05 U so per 1 g of the adipose tissue.
[7] The method of any one of [1] to [6], wherein the collagenase is derived from Clostridium histolyticum.
[8] The method of any one of [1] to [7], wherein the neutral protease is derived from Clostridium histolyticum.
[9] The method of any one of [1] to [8], wherein the enzyme solution further comprises clostripain and/or thermolysin.
[10] The method of any one of [1] to [8], wherein the enzyme solution is free of clostripain or thermolysin.
[11] The method of any one of [1] to [10], wherein the adipose tissue is a human adipose tissue.
[12] A method for preparing a cell population comprising a vascular endothelial cell and a vascular endothelial progenitor cell derived from an adipose tissue, the method comprising the following step (2):

(2) a step of enriching and/or proliferating the vascular endothelial cell and the vascular endothelial progenitor cell in the stromal vascular fraction obtained by the method of any one of [1] to [11].

[13] An enzyme preparation for dispersing adipose tissues, comprising a collagenase and a neutral protease, and showing not less than 1 U neutral protease activity with respect to 10,000 U collagenase activity.
[14] The enzyme preparation of [13], wherein the neutral protease activity is not less than 2 U with respect to 10,000 U collagenase activity.
[15] The enzyme preparation of [13], wherein the neutral protease activity is not less than 2.5 U with respect to 10,000 U collagenase activity.
[16] The enzyme preparation of [13], wherein the enzyme solution has a collagenase:neutral protease activity ratio of from 34,000:9 to 34,000:45.
[17] The enzyme preparation of any one of [13] to [16], wherein the collagenase is derived from Clostridium histolyticum.
[18] The enzyme preparation of any one of [13] to [17], wherein the neutral protease is derived from Clostridium histolyticum.
[19] The enzyme preparation of any one of [13] to [18], further comprising clostripain and/or thermolysin.
[20] The enzyme preparation of any one of [13] to [18], which is free of clostripain or thermolysin.

### DESCRIPTION OF EMBODIMENTS

### 1. Method for Preparing Stromal Vascular Fraction (SVF)

The first aspect of the present invention relates to a method for preparing a stromal vascular fraction (SVF) from an adipose tissue (hereinafter to be referred to as the “SVF preparation method”). In the SVF preparation method of the present invention, the following step (1) is performed:

(1) a step of treating an adipose tissue with an enzyme solution comprising a collagenase and a neutral protease, and showing not less than 1 U neutral protease activity with respect to 10,000 U collagenase activity, and recovering cells.

Adipose tissue can be collected from human, mammals other than human (including pet animals, domestic animals, experiment animals, specifically, for example, mouse, rat, guinea pig, hamster, monkey, bovine, swine, goat, sheep, dog, cat, etc.), birds (chicken, quail, etc.), and the like by a means such as excising, aspiration, or the like. Examples of the adipose tissue include subcutaneous fat, visceral fat, intramuscular fat, and intermuscular fat. Adipose tissue can also be obtained by aspiration from a cannula intubated into the abdomen, femoral, breech, or systemic subcutaneous adipose tissue. The amount of the obtained adipose tissue is, for example, 1 g to 1000 g, preferably 1 g to 500 g, more preferably 1 g to 100 g, further preferably 2 g to 50 g, and further more preferably 2 g to 40 g, but the amount is not limited to these. Subcutaneous fat is particularly preferred since it can be collected very easily under, for example, topical anesthesia, and causes only a small burden on the donor at the time of collection. Generally, one kind of adipose tissue is used, but two or more kinds of adipose tissue can also be used in combination. In addition, adipose tissue (may not be allogenic adipose tissue) collected multiple times may be mixed and used in the following operations.

The collected adipose tissue is subjected where necessary to removal of the blood components attached thereto (e.g., removal of blood components by washing adipose tissue with suitable buffer or culture medium) and mincing, and used for the following enzyme treatments. When an aspirated adipose tissue is used, the aspirated adipose tissue is preferably allowed to stand to separate the fat layer from the aqueous layer. In addition, the fat layer and the aqueous layer can also be separated by processing the aspirated adipose tissue with a centrifuge. After separation of the fat layer and the aqueous layer, the aqueous layer is recovered and removed, whereby the fat layer can be isolated. The obtained adipose tissue may be subjected to an enzyme treatment after washing with, for example, saline or the like. It is preferable to warm the adipose tissue before being subjected to an enzyme treatment, in a water bath at room temperature or around 37° C. for about 5 to 15 min.

Adipose tissue is subjected to an enzymatic treatment (enzymatic reaction). In the present invention, the yield of SVF is improved by using collagenase and neutral protease in combination in the enzyme treatment, and increasing the content percentage of the neutral protease in the enzyme solution (activity ratio with collagenase). Specifically, an enzyme solution containing collagenase and a neutral protease and having a neutral protease activity of 1 U or more with respect to 10,000 U collagenase activity is prepared, and adipose tissue is treated with the enzyme solution. The enzyme solution used in the present invention may be prepared, for example, by dissolving or diluting an enzyme preparation prepared so that collagenase and neutral protease each have desired activities.

Preferably, in order to increase the yield of SVF, an enzyme solution having a higher neutral protease activity with respect to collagenase activity (hereinafter to be referred to as “activity ratio” for convenience of explanation) is used. Specifically, in a preferred embodiment, an enzyme solution having a neutral protease activity of not less than 2 U (e.g., in the range of 2 U to 50 U) with respect to collagenase activity 10000 U is used, in a more preferred embodiment, an enzyme solution having a neutral protease activity of not less than 2.5 U (e.g., in the range of 2.5 U to 50 U) with respect to collagenase activity 10000 U is used, in a further preferred embodiment, an enzyme solution having a neutral protease activity of not less than 3 U (e.g., in the range of 3 U to 50 U) with respect to collagenase activity 10000 U is used, and in a further more preferred embodiment, an enzyme solution having a neutral protease activity of not less than 5 U (e.g., in the range of 5 U to 50 U) with respect to collagenase activity 10000 U is used. Specific examples of particularly preferred activity ratio can include 34,000:9 to 34,000:45. The activities of collagenase and neutral protease are calculated by the measurement method shown in the section of Examples described later.

In order to treat the adipose tissue with the enzyme solution, for example, the enzyme solution is added to the adipose tissue or the adipose tissue is immersed in the enzyme solution to form a state in which the enzyme in the enzyme solution can contact (act on) the adipose tissue. Under this state, the tissue is treated in the enzyme solution under conditions where the enzyme can react, that is, the enzyme is allowed to react. The conditions of the enzyme reaction are not particularly limited as long as collagenase and neutral protease show activity and the cells are separated from the adipose tissue. For example, the pH is set to 5 to 10, preferably 6 to 9, the temperature is set to, for example, 25° C. to 50° C., preferably 30° C. to 45° C., further preferably 35° C. to 40° C. (specific example is 37° C.), and the reaction time is set to, for example, 10 min to 3 hr, preferably 15 min to 1 hr (specific examples are 20 min, 30 min, 40 min, 50 min). For efficient progress of the enzyme reaction, it is advisable to shake the reaction vessel (linear shaking, rotational shaking, etc.).

The enzyme solution used in the present invention shows a higher content percentage of neutral protease than when using a crude collagenase preparation (e.g., “collagenase”, Wako). While collagenase plays an important role in dispersing adipose tissue in the present invention, the high content percentage of neutral protease affects the yield of SVF and the number of vascular endothelium-related cells (vascular endothelial cell/vascular endothelial progenitor cell) in SVF. According to the present invention, the yield of SVF is improved. Typically, the number of vascular endothelium-related cells in SVF is also increased. Therefore, the present invention is extremely effective as a means for efficiently obtaining vascular endothelium-related cells derived from adipose tissue (the method for preparing vascular endothelium-related cells is described in the second aspect below).

The amounts of the collagenase and neutral protease in the enzyme solution are not particularly limited as long as the cells can be separated from the adipose tissue. For example, they are contained in such amounts that afford the activity value of not less than 500 U (e.g., 500 to 30,000 U) for collagenase and not less than 0.05 U (e.g., 0.05 to 20 U) for neutral protease, preferably 1,000 to 20,000 U for collagenase and 0.1 to 15 U for neutral protease, more preferably 3,000 to 10,000 U for collagenase and 0.15 to 10 U for neutral protease, each per 1 g of the adipose tissue (activity ratio of collagenase and neutral protease in the enzyme solution is as mentioned above).

The origin of collagenase or neutral protease is not particularly limited as long as they are useful for the separation of cells from the adipose tissue. For example, a collagenase derived from Clostridium histolyticum and a neutral protease derived from Clostridium histolyticum can be used. These collagenases and neutral proteases derived from the microorganisms can be prepared by separating and purifying the culture medium or bacterial cells of the microorganisms that produce them (producing strains). A host microorganism into which a collagenase gene (or a gene obtained by modifying the gene) obtained from a collagenase-producing strain has been introduced can also be used as a collagenase-producing strain. The same applies to the neutral protease. For separation and purification of collagenase and neutral protease, various chromatographies (ion exchange chromatography, hydrophobic chromatography, affinity chromatography, etc.), salting out and the like can be used. For preparation of neutral protease, a literature “Dendo M, et al. Synergistic effect of neutral protease and clostripain on pancreatic islet isolation. Transplantation. In press, 2015” can be referred to. Note that Amano Enzyme Inc. (e.g., Collagenase “Amano” GMP), Worthington Biochemical Corporation (e.g., Collagenase, Purified), Vitacyte LLC (e.g., Collagenase HA, Collagenase MA, rCollagenase HI), Roche (e.g., Collagenase A), and the like provide purified collagenases, and collagenase to be used in the present invention can be easily obtained.

Preferably, a collagenase derived from Clostridium histolyticum and a neutral protease derived from Clostridium histolyticum are used. The collagenase derived from Clostridium histolyticum has a wide range of substrate specificities, characteristically acts on almost all types of collagen, and is particularly useful for dispersing adipose tissue. Similarly, the neutral protease derived from Clostridium histolyticum is specific to the FAGFYA substrate, characteristically has weak cytotoxicity, and is particularly useful for dispersing adipose tissue.

For stabilization and activation of collagenase, it is preferable to contain Ca²⁺ in the enzyme solution. Therefore, it is preferable to add, for example, CaCl₂ to the enzyme solution. When CaCl₂ is added, its concentration in the enzyme solution is, for example, 1 mM to 10 mM, preferably 2 mM to 5 mM, further preferably 2 mM to 4 mM.

In one preferred embodiment of the invention, the enzyme solution is free of clostripain and thermolysin. That is, only collagenase and neutral protease are substantially contained as enzymes for disintegrating adipose tissue. This embodiment is also advantageous in that the composition of the enzyme solution is simplified and the preparation of the enzyme solution is easy. In addition, the absence of clostripain in the enzyme solution is preferable particularly because the number of vascular endothelium-related cells in SVF increases.

On the other hand, clostripain or thermolysin, or both of them may be contained in an enzyme solution, and the action of these enzymes may be utilized to further improve the dispersion efficiency of adipose tissue and the yield of SVF. In this case, as a content of clostripain, for example, the clostripain activity is, for example, more than 0 U to 2,000 U, preferably 1 U to 1,500 U, more preferably 10 U to 1,100 U, with respect to 10,000 U collagenase activity. Too much clostripain content affects the SVF yield. On the other hand, as a content of thermolysin, for example, the thermolysin activity is, for example, more than 0 U to 10,000 U, preferably 1 U to 7,000 U, more preferably 10 U to 5,000 U, with respect to 10,000 U collagenase activity. Too much thermolysin content affects the SVF yield. The origin of clostripain or thermolysin is not particularly limited and, for example, clostripain derived from Clostridium histolyticum, and thermolysin derived from Bacillus thermoproteolyticus or Geobacillus stearothermophilus can be used. The clostripain and thermolysin derived from these microorganisms can be prepared by separation and purification of the culture medium or bacterial cells of the microorganisms (producing strains) that produce them, or by genetic engineering techniques, as in the case of collagenase. The methods of the separation and purification are also the same as those in the case of collagenase. For the preparation of clostripain, the above-mentioned document “Dendo M, et al. Synergistic effect of neutral protease and clostripain on pancreatic islet isolation. Transplantation. In press, 2015” can be referred to.

Enzymes other than the above-mentioned enzymes may be contained in the enzyme solution, as long as it does not affect the action of each of the above-mentioned enzymes (collagenase, neutral protease, clostripain, thermolysin) and the effect thereof (dispersion of adipose tissue).

The cell population obtained by the enzyme treatment contains multipotent stem cells, vascular endothelial cells, stromal cells, blood cells, and the like. Generally, the sediment (cell pellets) obtained by a centrifugation treatment is recovered as SVF. The conditions for the centrifugation treatment vary depending on the type and amount of cells, and are, for example, 1 to 20 minutes, 500 G to 1000 G. It is preferable to perform a filter treatment (a cell strainer or the like can be used) or the like to remove the enzyme-undigested tissue and the like prior to the centrifugation treatment. In addition, the cells obtained by the centrifugation treatment may be subjected to a filter treatment or the like to remove unnecessary components. Furthermore, a hemolysis treatment may be performed before or after the centrifugation treatment. In the present specification, the cell population means a population containing a large number of one or more types of cells.

The type, ratio, and the like of the cell population constituting SVF depend on the origin and type of adipose tissue used, enzyme treatment conditions, and the like. Generally, SVF fraction consists of cells (CD45-negative) derived from adipose tissue and cells (CD45-positive) derived from peripheral blood, and the cells (CD45-negative) derived from adipose tissue contain vascular endothelial cell/vascular endothelial progenitor cells constituting a CD34-positive and CD31-positive cell population (CD45⁻ CD34⁺ CD31⁺), and ASC (adipose tissue-derived stromal cells/adipose tissue-derived stem cells) constituting a CD34-positive and CD31-negative cell population (CD45⁻ CD34⁺ CD31⁺).

### 2. Method for Preparing Vascular Endothelium-Related Cells

In the second aspect, the present invention provides, a method for preparing a cell population containing vascular endothelium-related cells (i.e., vascular endothelial cells and vascular endothelial progenitor cells) derived from adipose tissue, from SVF obtained by the SVF preparation method of the present invention (hereinafter to be referred to as “vascular endothelium-related cell preparation method”). In the vascular endothelium-related cell preparation method of the present invention, the SVF obtained by the SVF preparation method of the present invention is used to obtain a population with high percentage (purity) of the vascular endothelium-related cells, i.e., the vascular endothelium-related cell population with high purity. In a high purity vascular endothelium-related cell population, the percentage of the number of vascular endothelium-related cells in the cell population, namely, “(vascular endothelium-related cells/cell number of whole cell population)×100(%)”, is, for example, not less than 50%, preferably not less than 60%, more preferably not less than 70%, further preferably not less than 80%, further more preferably not less than 90%, still more preferably not less than 95%. The number of vascular endothelial cells in SVF is generally about one to several %. Therefore, according to the vascular endothelium-related cell preparation method of the present invention, the purity of the vascular endothelium-related cells is strikingly increased, and a cell population containing vascular endothelium-related cells at a far higher concentration (high purity) than in SVF can be obtained. The vascular endothelium-related cell can be identified as a CD45-negative and CD31-positive cell. In addition, markers such as CD144 and CD146 can also be used to identify vascular endothelium-related cells.

Vascular endothelium-related cells can be used for the treatment of ischemic diseases of all organs through angiogenesis of blood vessels, and are necessary for regenerating organs outside or inside the body, together with organ-constituting cells, organ-specific progenitor cells, stem cells (including embryonic stem cells and iPS cells), and cells induced from stem cells, for the regenerative medicine for organs (organs, tissues). The cell population obtained by the vascular endothelium-related cell preparation method of the present invention is useful as a valuable cell pharmaceutical product that can be used to treat a wide range of diseases.

In the vascular endothelium-related cell preparation method of the present invention, a cell population containing high purity vascular endothelium-related cells is obtained by selective recovery, proliferation, and the like of the vascular endothelium-related cells in SVF. Typically, the following step (2) is performed.

(2) a step of enriching and/or proliferating the vascular endothelial cells and the vascular endothelial progenitor cells in the stromal vascular fraction obtained by the SVF preparation method of the present invention.

Generally, vascular endothelium-related cells in SVF are collectively enriched or proliferated, but vascular endothelial cells or vascular endothelial progenitor cells may be targeted for enrichment or proliferation. Various methods can be adopted for enrichment or proliferation. Not only a known method but also a method developed hereafter may be used. A typical operation for enrichment is sorting (selection and recovery) by cell markers. For example, cell markers such as CD45 and CD31, which are useful for selecting vascular endothelium-related cells, can be used. The enrichment operation may be performed a plurality of times. For example, cells enriched with a specific marker are cultured and proliferated, and then enriched with another marker. Such a series of operations may be repeated, in which case the markers used for each operation may be the same or different. In addition, the cell markers may be used alone or two or more markers may be used in combination. For example, the cell markers CD45 and CD31 are used for the first sorting, and after sorting and recovering CD45-negative and CD31-positive cells, the recovered cells are cultured and proliferated, and then the CD31-positive cells are sorted and recovered using the cell marker CD31.

For example, magnetic cell separation method (MACS), FACS, or the like can be used for cell sorting and recovery using a cell marker. According to MACS, antibodies against marker proteins can be immobilized on magnetic beads and powerful magnets can be used to separate the cells of interest on the inner wall of a cylindrical container (column) or simply in a tube. As the magnetic bead reagent to be immobilized, general ones such as MACS (manufactured by Miltenyi Biotec) and IMag (manufactured by BD Japan) can be used. In FACS, if a flow cytometer having a cell sorter function is used, only specific cells that emit a specified fluorescence can be separated. Examples of such a device include FACSAriaII (manufactured by Japan BD), JSAN (manufactured by Bay bioscience Co., Ltd.), MoFlo XDP (manufactured by BECKMAN COULTER), and the like.

Vascular endothelium-related cells can be proliferated by a conventional method. That is, vascular endothelium-related cells may be incubated in a medium suitable for culturing thereof under appropriate conditions (e.g., 37° C., in CO₂ incubator). The culture period is not particularly limited, and is, for example, 1 to 21 days. The cells may be passaged in the middle of culturing. The number of passages is not particularly limited. In addition, the medium may be exchanged, for example, every 1 to 2 days. As the medium, for example, EGM-2 (Lonza), αMEM, Dulbecco's modified Eagle medium (DMEM), Dulbecco's modified Eagle medium/Ham F-12 mixed medium (DMEM/F12), RPMI1640, and the like can be used. Preferred media are, for example, EGM-2 medium (Lonza) and EGM-2 MV (Lonza). Various additives used in general cell culture, such as serum, various vitamins, various antibiotics, various hormones, and various growth factors, may be added to the medium.

As step (2), two methods (referred to as the first method and the second method) disclosed in JP-A-2019-88279 may be used. In the first method, a cell population containing CD31-positive cells is obtained by sorting CD31-positive cells from SVF (CD45-negative and CD31-positive cells may be sorted, or only CD31-positive cells may be sorted), and then the cell population is cultured for 1 hr to 7 days, and CD31-positive cells are sorted from the cell population obtained by the culture, whereby a cell population containing CD31-positive cells is obtained. On the other hand, in the second method, a cell population containing CD45-negative and CD31-positive cells is obtained by sorting CD45-negative and CD31-positive cells from SVF, the cell population is cultured for 2 to 6 days (or 3 to 6 days), and CD31-positive cells are sorted from the cell population obtained by the culture, whereby a cell population containing CD31-positive cells is obtained. The SVF may be cultured for 1 hr to 5 days (or 1 to 4 days) and then subjected to the first method or the second method. For details of the above-mentioned first method and the second method, JP-A-2019-88279 may be referred to.

### 3. Enzyme Preparation

A further aspect of the present invention relates to an enzyme preparation for dispersing an adipose tissue. The enzyme preparation of the present invention is typically used for the above-mentioned SVF preparation method and vascular endothelium-related cell preparation method of the present invention. That is, it is used for preparing an enzyme solution to be used for treating an adipose tissue. Therefore, it characteristically contains collagenase and neutral protease, and shows neutral protease activity of not less than 1 U with respect to 10,000 U collagenase activity. The neutral protease activity is preferably not less than 2 U (e.g., in the range of 2 U to 50 U) with respect to 10,000 U collagenase activity, more preferably not less than 2.5 U (e.g., in the range of 2.5 U to 50 U) with respect to 10,000 U collagenase activity, further preferably not less than 3 U (e.g., in the range of 3 U to 50 U) with respect to 10,000 U collagenase activity, and further more preferably not less than 5 U (e.g., in the range of 5 U to 50 U) with respect to 10,000 U collagenase activity. Specific example of a particularly preferable activity ratio is 34,000:9 to 34,000:45. The contents of collagenase and neutral protease are not particularly limited and, for example, collagenase is contained at 1,000 U/g to 6,000,000 U/g per 1 g of the enzyme preparation, and neutral protease is contained at 0.1 U/g to 9,000 U/g per 1 g of the enzyme preparation (the ratio of collagenase and neutral protease in an enzyme preparation is as mentioned above).

The collagenase and neutral protease are not particularly limited and, for example, a collagenase derived from Clostridium histolyticum and a neutral protease derived from Clostridium histolyticum are used.

In one preferred embodiment, the enzyme preparation is free of clostripain and thermolysin. That is, only collagenase and neutral protease are substantially contained as enzymes for dispersing an adipose tissue. On the other hand, clostripain or thermolysin, or both of them may be contained in an enzyme preparation, and the action of these enzymes may be utilized to further improve the dispersion efficiency of adipose tissue and the yield of SVF. In this case, the content of clostripain is, for example, more than 0 U/g to 100,000 U/g per 1 g of the enzyme preparation, preferably 10 U/g to 50,000 U/g per 1 g of the enzyme preparation, and the content of thermolysin is, for example, more than 0 U/g to 5,000,000 U/g per 1 g of the enzyme preparation, preferably 10 U/g to 1,000,000 U/g per 1 g of the enzyme preparation.

Enzymes other than the above-mentioned enzymes may be contained in the enzyme preparation, as long as it does not affect the action of each of the above-mentioned enzymes (collagenase, neutral protease, clostripain, thermolysin) and the effect thereof (dispersion of adipose tissue).

The enzyme preparation may contain excipient, buffering agent, suspending agent, stabilizer, preservative, antiseptic, surfactant, saline, and the like besides the active ingredient (i.e., respective enzymes useful for dispersing adipose tissues). As the excipient, lactose, sorbitol, D-mannitol, maltodextrin, trehalose, sucrose, and the like can be used. As the buffering agent, Good's buffer (HEPES, etc.), phosphate, citrate, acetate, and the like can be used. As the stabilizer, propylene glycol, ascorbic acid, sodium chloride, calcium chloride, and the like can be used. As the preservative, phenol, benzalkonium chloride, benzyl alcohol, chlorobutanol, methylparaben, and the like can be used. As the antiseptic, benzalkonium chloride, p-hydroxybenzoic acid, chlorobutanol, and the like can be used. As the surfactant, poloxamer, and the like can be used.

The form of the enzyme preparation may be liquid or solid (including powder). In the latter case, it is typically prepared by powdering an enzyme solution containing the necessary components (each enzyme as active ingredient and other components added as needed) by freeze-drying, vacuum-drying, spray-drying, or the like.

### EXAMPLE

### 1. Activity Measurement/Activity Definition

### (1) Measurement Method and Activity Definition of Collagenase Activity

Collagenase activity is measured using Molecular Probes EnzChek Gelatinase/Collagenase Assay Kit (Invitrogen). Water (1 mL) is added to one DQ gelatin vial (manufactured by Molecular Probes) to make a 1 mg/mL solution. This solution is diluted 40-fold with 0.05 mol/L tris buffer (pH 7.6)-containing NaCl·CaCl₂ to give a 25 μg/mL substrate solution. An enzyme whose enzyme activity is already known is diluted with 0.05 mol/L tris buffer (pH 7.6)-containing NaCl₂·CaCl₂ to prepare a 0.1-0.4 U/mL solution. A sample is diluted with 0.05 mol/L tris buffer (pH 7.6)-containing NaCl₂·CaCl₂ to give a sample solution. The substrate solution (100 μL) is added to 100 μL of each of these solutions, the mixture is shaken, and the fluorescence is measured (excitation wavelength 485 nm, fluorescence wavelength 528 nm) every minute at room temperature for 1 hr. A calibration curve is drawn by plotting the fluorescence on the vertical axis and the enzyme concentration (0, 0.1, 0.2, 0.3, 0.4 U/mL) on the horizontal axis. The amount of enzyme that produces 1 μmol of L-leucine from collagen in 5 hr under 37° C., pH 7.5 conditions is taken as 1 unit (1U), and the enzyme activity is calculated from the following formula.

collagenase activity(U/g,mL)={(AT−AB)−b}/a×n

AB: amount of fluorescence of reaction solution at the start of reaction

AT: amount of fluorescence of reaction solution 1 hr later

a: slope of calibration curve obtained by calibration curve stock solution

b: y-section of calibration curve obtained by calibration curve stock solution

n: dilution fold

### (2) Measurement Method and Activity Definition of Clostripain Activity

A substrate solution (3 mL, 0.76 mmol/L N-Benzoyl-L-arginine ethyl ester hydrochloride, 0.4 mmol/L calcium chloride, 0.1 mol/L monopotassium phosphate/dipotassium phosphate buffer (pH 7.6)) is added to a quartz cell. After allowing same to stand at 25° C. for 5 min, an enzyme solution diluted to an appropriate concentration (0.0025 mol/L MOPS buffer (pH 7.4), 0.001 mol/L calcium chloride, 0.05 mL) is added, and the mixture is shaken immediately. While keeping this solution at 25° C., the absorbance (AT) at a wavelength of 255 nm is measured every 10 seconds for 5 min. The amount of enzyme that produces 1 μmol of N-Benzoyl-L-arginine per minute under this condition is taken as 1 unit (1 U), and the enzyme activity is calculated from the following formula.

Proteolytic activity(U/g)=(AT/min−AB/min)/0.81×3.05/0.05×n

AT/min: amount of change in absorbance per minute of the reaction solution

AB/min: amount of change in absorbance per minute of blank solution

0.81: millimolar absorption coefficient of N-Benzoyl-L-arginine at wavelength 255 nm

3.05: liquid volume (mL) at the time of reaction

0.05: liquid volume (mL) of sample added during enzymatic reaction

N: dilution fold

### (3) Measurement Method and Activity Definition of Neutral Protease (NP) Activity

A substrate solution (2.88 mL, 0.4 mmol/L N-[3-(2-Furyl)acryloyl]-Gly-Phe-Tyr-amide, 10% dimethyl sulfoxide, 0.1 mol/L tris buffer (pH 7.5)) is added to a quartz cell. After allowing same to stand at 37±0.5° C. for 5 min, an enzyme solution (0.12 mL) diluted to an appropriate concentration is added and mixed. While keeping this solution at 37° C., the absorbance AT at a wavelength of 344 nm is measured every 10 seconds for 100 seconds. The amount of enzyme that produces 1 μmol of N-[3-(2-Furyl)acryloyl]-Gly per minute under this condition is taken as 1 unit (1 U), and the enzyme activity is calculated from the following formula.

Proteolytic activity (U/g)=−{(AT80−AB80)−(AT20−AB20)}/0.524×3000/120×n

AT20: absorbance of reaction solution at 20 seconds

AT80: absorbance of reaction solution at 80 seconds

AB20: absorbance of blank solution at 20 seconds

AB80: absorbance of blank solution at 80 seconds

0.524: millimolar absorption coefficient of FAGFYA at wavelength 344 nm

3000: liquid volume (μL) at the time of reaction

120: liquid volume (μL) of sample added at the time of enzyme reaction

n: dilution fold

### (4) Measurement Method and Activity Definition of Thermolysin Activity

An enzyme solution (1 mL) is added to and mixed with a substrate solution (0.6% W/V 2-amino-2-hydroxymethyl-1,3-propanediol, 0.7% W (dry weight)/V milk casein, pH 7.0) (a casein solution (5 mL)) preheated at 37° C. After 30 min at 37° C., 0.11 mol/L trichloroacetic acid test solution (5 mL) is added to discontinue the reaction. After allowing to stand at 37° C. for 30 min, the mixture is mixed well and filtered with 11 cm Whatman No. 42 filter paper. The absorbance of the filtrate at a wavelength of 275 nm is measured. The amount of enzyme that liberates a substance corresponding to the absorbance of 1.5 μg of L-tyrosine in 1 min under this condition is defined as 1 proteolytic activity unit (1 U), and the enzyme activity is calculated from the following formula.

proteolytic activity (U/g)=(A30−A0)/As×11/30×n

A30: absorbance of enzyme reaction solution

A0: absorbance of blank solution

As: tyrosine amount (μg) when absorbance difference obtained from tyrosine calibration curve is 1

11: final volume (mL) of reaction solution

30: reaction time (min)

n: dilution fold per 1 g enzyme

### 2. Preparation Method of SVF Nucleated Cell

Adipose tissue is divided by about 10 mL in a 50 mL centrifugation tube, and the volume and mass are measured. HBSS buffer (pH 6.7-7.8) in which a predetermined amount of the enzyme to be used is dissolved is mixed with adipose tissue in an approximately equal amount. A parafilm is wrapped around the lid of the tube and the mixture is reacted (dispersing adipose tissue) by shaking at 120 rpm, 37° C. for 20 min. A 2-fold amount of HBSS buffer previously cooled at 4° C. is added to the adipose tissue to terminate the enzyme reaction. After centrifugation at 800 g for 10 min, and the tissue residue/supernatant is removed with a pipette, leaving 2 to 3 mL. An appropriate amount of HBSS buffer previously cooled at 4° C. is added, and the cell suspension is passed through a cell strainer (mesh size: 100 μm for the first time, 40 μm for the second time). After centrifugation at 800 g for 10 min, the supernatant was removed, and the cells were suspended in 2 mL of DMEM/F12 medium (Wako) to give an SVF nucleated cell suspension.

### 3. Measurement of Numbers of SVF Nucleated Cells and Vascular Endothelium-Related Cells (EC and EPC)

### (1) Measurement Method of Number of Nucleated Cells in SVF

The number of SVF nucleated cells in the SVF nucleated cell suspension was measured by Luna-stem (Logos Biosystems).

### (2) Measurement Method of Number of Vascular Endothelium-Related Cells in SVF

The number of vascular endothelium-related cells in SVF was measure by the FACS (Fluorescence activated cell sorting) method (see Patent Literature 1).

### 4. Examination of Combination of Clostripain (CP) and Neutral Protease (NP)

The number of SVF nucleated cells when CP and/or NP were/was used in combination with collagenase (CL) during dispersing adipose tissue was compared and examined. The following test groups different in the enzymes to be used were prepared, and SVF nucleated cell suspensions were prepared by the above-mentioned method. The number of SVF nucleated cells in the SVF nucleated cell suspension was measured and evaluated by the ratio to the number of SVF nucleated cells with the use of 0.2% (w/v) of Wako CL (product name: collagenase) as 1.

test group 1: combined use of Clostridium histolyticum-derived purified CL (34,000 U), Clostridium histolyticum-derived purified CP (3,600 U), and Clostridium histolyticum-derived purified NP (9 U)

test group 2: combined use of Clostridium histolyticum-derived purified CL (34,000 U) and Clostridium histolyticum-derived purified CP (3,600 U)

test group 3: combined use of Clostridium histolyticum-derived purified CL (34,000 U) and Clostridium histolyticum-derived purified NP (9 U)

test group 4: Clostridium histolyticum-derived purified CL (34,000 U)

The experiment results are shown in Table 1. Compared with the case of using only CL (test group 4), the yield of SVF nucleated cells increased by the combined use of CP and NP (test groups 1 to 3). In particular, the SVF yield greatly increased when used in combination with NP (test group 3). It was found that an increase in the SVF yield was small by the combined use of CL and CP (test group 3), but the SVF yield significantly increased when NP was further used in combination (test group 1).

TABLE 1

| | purified CL + purified | | | purified |
|---|---|---|---|---|
| | CP/purified NP | | | CL |
| collagenase (CL) activity | 34,000 | 34,000 | 34,000 | 34,000 |
| (Enzchek kit) | | | | |
| clostripain (CP) (BAEE | 3,600 | 3,600 | — | — |
| method) | | | | |
| neutral protease (NP) | 9.0 | — | 9.0 | — |
| activity (FAGFYA | | | | |
| method) | | | | |
| SVF nucleated cell | 2.3 | 1.2 | 1.6 | 0.4 |
| number (ratio to Wako | | | | |
| CL (0.2%)) | | | | |

### 4. Examination of Amount of Clostripain (CP) Used

The relationship between the amount of CP used and the number of SVF nucleated cells when CP was used during dispersing adipose tissue (combined use of collagenase (CL)) was studied. The following test groups different in the kind and amount of use of enzymes were prepared, and SVF nucleated cell suspensions were prepared by the above-mentioned method. The number of SVF nucleated cells in the SVF nucleated cell suspension was measured and evaluated by the ratio to the number of SVF nucleated cells with the use of 0.2% (w/v) of Wako CL (product name: collagenase) as 1.

test group 1: 0.0067% (w/v) of Wako CL

test group 2: combined use of Clostridium histolyticum-derived purified CL (34,000 U) and Clostridium histolyticum-derived purified CP (360 U)

test group 3: combined use of Clostridium histolyticum-derived purified CL (34,000 U) and Clostridium histolyticum-derived purified CP (3,600 U)

test group 4: combined use of Clostridium histolyticum-derived purified CL (34,000 U) and Clostridium histolyticum-derived purified CP (18,000 U)

The experiment results are shown in Table 2. When the amount of CL used was reduced from 0.2% to 0.067% (test group 1), the yield of SVF nucleated cells decreased by about 10%. On the other hand, when CP was singly used in combination with CL, the SVF yield did not increase even if the amount of CP was increased (test groups 2 to 4). That is, it was found that the amount of CP does not have a direct effect on the number of SVF nucleated cells.

TABLE 2

| | Wako CL | | | |
|---|---|---|---|---|
| | (0.067%) | purified CL + purified CP | | |
| collagenase (CL) activity | 34,000 | 34,000 | 34,000 | 34,000 |
| (Enzchek kit) | | | | |
| clostripain (CP) activity | 1,200 | 360 | 3,600 | 18,000 |
| (BAEE method) | | | | |
| neutral protease (NP) | 3.0 | — | — | — |
| activity (FAGFYA | | | | |
| method) | | | | |
| SVF nucleated cell | 0.87 | 0.89 | 0.74 | 0.75 |
| number (ratio to Wako | | | | |
| CL (0.2%)) | | | | |

### 5. Examination of Amount of Neutral Protease (NP) Used

The relationship between the amount of NP used and the number of SVF nucleated cells when NP was used during dispersing adipose tissue (combined use of collagenase (CL)) was studied. The following test groups different in the kind and amount of use of enzymes were prepared, and SVF nucleated cell suspensions were prepared by the above-mentioned method. The number of SVF nucleated cells in the SVF nucleated cell suspension was measured and evaluated by the ratio to the number of SVF nucleated cells with the use of 0.2% (w/v) of Wako CL (product name: collagenase) as 1.

test group 1: 0.0067% (w/v) of Wako CL

test group 2: combined use of Clostridium histolyticum-derived purified CL (34,000 U) and Clostridium histolyticum-derived purified NP (0.9 U)

test group 3: combined use of Clostridium histolyticum-derived purified CL (34,000 U) and Clostridium histolyticum-derived purified NP (9 U)

test group 4: combined use of Clostridium histolyticum-derived purified CL (34,000 U) and Clostridium histolyticum-derived purified NP (45 U)

The experiment results are shown in Table 3. Even when NP was singly used in combination with CL, the SVF yield increased when the amount of NP was increased. When the NP activity was set to not less than 9, the SVF yield was greatly improved as compared with the case where Wako CL was used. Thus, it was clarified that the combined use of CL and purified NP is effective for efficient preparation of SVF, and that the SVF yield is improved by increasing the amount of purified NP.

TABLE 3

| | Wako CL | | | |
|---|---|---|---|---|
| | (0.067%) | purified CL + purified NP | | |
| collagenase (CL) activity | 34,000 | 34,000 | 34,000 | 34,000 |
| (Enzchek kit) | | | | |
| clostripain (CP) activity | 1,200 | — | — | — |
| (BAEE method) | | | | |
| neutral protease (NP) | 3.0 | 0.9 | 9.0 | 45 |
| activity (FAGFYA | | | | |
| method) | | | | |
| SVF nucleated cell | 0.87 | 0.98 | 1.7 | 1.9 |
| number (ratio to Wako | | | | |
| CL (0.2%)) | | | | |

### 6. Examination of Effect and Amount of Thermolysin (TP) Used

The relationship between the amount of TP used and the number of SVF nucleated cells when TP was used during dispersing adipose tissue (combined use of collagenase (CL)) was studied. The following test groups different in the amount of use of enzymes were prepared, and SVF nucleated cell suspensions were prepared by the above-mentioned method. The number of SVF nucleated cells in the SVF nucleated cell suspension was measured and evaluated by the ratio to the number of SVF nucleated cells with the use of 0.2% (w/v) of Wako CL (product name: collagenase) as 1.

test group 1: combined use of Clostridium histolyticum-derived purified CL (69,000 U) and TP (6,000 U) of Thermolysine “Amano” GMP

test group 2: combined use of Clostridium histolyticum-derived purified CL (69,000 U) and TP (9,000 U) of Thermolysine “Amano” GMP

test group 3: combined use of Clostridium histolyticum-derived purified CL (69,000 U) and TP (12,000 U) of Thermolysine “Amano” GMP

The experiment results are shown in Table 4. The SVF yield decreased when the amount of TP was increased. Thus, it was clarified that even though TP can be used for the preparation of SVF, the use thereof in an amount more than necessary results in a decrease in the SVF yield.

TABLE 4

| | purified CL + purified TP | | |
|---|---|---|---|
| collagenase (CL) activity | 6,900 | 6,900 | 6,900 |
| (Enzchek kit) | | | |
| thermolysin (TP) (FCC) | 6000 | 9000 | 12000 |
| SVF nucleated cell number | 0.44 | 0.34 | 0.25 |
| (ratio to Wako CL (0.2%)) | | | |

### 7. Comparison of Number of Vascular Endothelium-Related Cells in the SVF

Whether the number of vascular endothelium-related cells in SVF changes by not using CP during dispersing adipose tissue was studied. SVF nucleated cell suspensions were prepared by the above-mentioned method. The number of vascular endothelium-related cells in the SVF was measured and evaluated by the ratio to the number of vascular endothelium-related cells in the SVF with the use of 0.2% (w/v) of Wako CL (product name: collagenase) as 1.

The experiment results are shown in Table 5. When adipose tissue was dispersed under the condition that did not contain CP (treatment with purified CL and purified NP), the number of vascular endothelium-related cells in the extracted SVF increased. That is, more vascular endothelium-related cells could be recovered. As described above, it was clarified that the combined use of purified CL and purified NP without using CP (CP is not contained in the enzyme solution) is effective as a means for efficiently preparing or recovering vascular endothelium-related cells from adipose tissue.

TABLE 5

| | Wako CL | purified CL + |
|---|---|---|
| | (0.2%) | purified NP |
| collagenase activity (Enzchek kit) | 100,000 | 100,000 |
| clostripain (CP) activity (BAEE method) | 3,600 | — |
| neutral protease (NP) activity (FAGFYA | 9 | 9 |
| method) | | |
| EC number (ratio to Wako product (0.2%)) | 1 | 1.6 |

### INDUSTRIAL APPLICABILITY

According to the present invention, SVF which is useful in regenerative medicine, research, and the like can be prepared efficiently. Therefore, the use and utilization of the present invention in the field of regenerative medicine is particularly expected.

The present invention is not limited in any way to the description of the embodiments and examples of the invention described above. The present invention also includes various modified embodiments as long as they can be easily conceived of by a person skilled in the art without departing from the spirit and scope of the attached CLAIMS. The contents disclosed in publications cited in the present specification, including papers, published unexamined patent applications, patent publications, and the like are hereby incorporated in their entireties by reference, to the extent that they have been disclosed herein.

This application is based on a patent application No. 2019-219192 filed in Japan (filing date: Dec. 4, 2019), the contents of which are incorporated by reference in full herein.

1. A method for preparing a stromal vascular fraction from an adipose tissue, the method comprising the following step (1):
(1) a step of treating an adipose tissue with an enzyme solution comprising a collagenase and a neutral protease, and showing not less than 1 U neutral protease activity with respect to 10,000 U collagenase activity, and recovering cells.

2. The method according to claim 1, wherein the neutral protease activity of the enzyme solution is not less than 2 U with respect to 10,000 U collagenase activity.

3. The method according to claim 1, wherein the neutral protease activity of the enzyme solution is not less than 2.5 U with respect to 10,000 U collagenase activity.

4. The method according to claim 1, wherein the enzyme solution has a collagenase:neutral protease activity ratio of from 34,000:9 to 34,000:45.

5. The method according to claim 1, wherein the enzyme solution has a collagenase content of not less than 500 U per 1 g of the adipose tissue.

6. The method according to claim 1, wherein the enzyme solution has a neutral protease content of not less than 0.05 U per 1 g of the adipose tissue.

7. The method according to claim 1, wherein the collagenase is derived from Clostridium histolyticum.

8. The method according to claim 1, wherein the neutral protease is derived from Clostridium histolyticum.

9. The method according to claim 1, wherein the enzyme solution further comprises clostripain and/or thermolysin.

10. The method according to claim 1, wherein the enzyme solution is free of clostripain or thermolysin.

11. The method according to claim 1, wherein the adipose tissue is a human adipose tissue.

12. A method for preparing a cell population comprising a vascular endothelial cell and a vascular endothelial progenitor cell derived from an adipose tissue, the method comprising the following step (2):
(2) a step of enriching and/or proliferating the vascular endothelial cell and the vascular endothelial progenitor cell in the stromal vascular fraction obtained by the method according to claim 1.

13-20. (canceled)

21. A method for dispersing adipose tissues, the method comprising the following step (1):
(1) a step of treating an adipose tissue with an enzyme solution comprising a collagenase and a neutral protease, and showing not less than 1 U neutral protease activity with respect to 10,000 U collagenase activity.

22. The method according to claim 21, wherein the neutral protease activity is not less than 2 U with respect to 10,000 U collagenase activity.

23. The method according to claim 21, wherein the neutral protease activity is not less than 2.5 U with respect to 10,000 U collagenase activity.

24. The method according to claim 21, wherein the enzyme solution has a collagenase:neutral protease activity ratio of from 34,000:9 to 34,000:45.

25. The method according to claim 21, wherein the collagenase is derived from Clostridium histolyticum.

26. The method according to claim 21, wherein the neutral protease is derived from Clostridium histolyticum.

27. The method according to claim 21, wherein the enzyme solution further comprises clostripain and/or thermolysin.

28. The method according to claim 21, wherein the enzyme solution is free of clostripain or thermolysin.
